(19) United States
(12) Patent Application Publication
(10) Pub. No.: US 2024/0090918 A1
(43) Pub. Date: Mar. 21, 2024

(54) Device, system, and method for self dry needle for muscular pain or discomfort

(71) Applicant: Dry Needling Innovation Systems, LLC, Holmen, WI (US)

(72) Inventor: Dana Halderson, Holmen, WI (US)

(21) Appl. No.: 18/367,897

(22) Filed: Sep. 13, 2023

Related U.S. Application Data
(60) Provisional application No. 63/407,931, filed on Sep. 19, 2022.

(51) Int. Cl. A61B 17/34

(52) U.S. Cl. CPC A61B 17/3403

(57) ABSTRACT

Device, system, and method for dry needling comprising a device having a surface area configured to surround a location on a user, one or more openings in the surface area, each opening supporting a directional needle guide configured to allow a needle to pass therethrough to the location on the user; and one or more needles configured to fit within each directional needle guide

### CROSS-REFERENCE TO RELATED APPLICATION

This application claims priority to and the benefit of U.S. provisional patent application Ser. No. 63/407,931, filed Sep. 19, 2022, the contents of which are incorporated herein in their entirety.

### BACKGROUND

Musculoskeletal pain results from repetitive movement or injury to a body part such as the forearm, shoulder, thigh, lower leg and/or foot. This causes pain and discomfort, and disrupts the activities of a person's daily life.

Dry needling is a method of relieving pain associated with repetitive movements and/or other injury. Generally, dry needling is carried out by physical therapists, chiropractors or other clinicians and is done in-office. Clinicians use dry needling to resolve musculoskeletal pain.

Because healthcare system clinicians currently perform dry needling, limited access to healthcare and high costs prevent many people from obtaining such services.

### SUMMARY

An aspect of the present disclosure relates to a system for dry needling comprising a device having a surface area configured to surround a location on a user, one or more openings in the surface area, each opening supporting a directional needle guide configured to allow a needle to pass therethrough to the location on the user; and one or more needles configured to fit within each directional needle guide.

The surface area comprises a flexible material, fabric, or plastic and is configured to removably surround the location on the user.

In one or more embodiments the surface area is a sleeve configured to surround an arm, leg, or other appendage of the user.

The sleeve is a pull-device of unitary construction or is a wrap sleeve that can be secured around the location of the user with closures of clasps, clips, hook and loop fastener or combinations thereof.

The openings in the surface area correspond to one or more muscle locations on the user such that when one of the one or more needles is inserted into each directional guide the needle is in a position to penetrate the user into the muscle tissue.

The user is able to self-administer the needles.

The sleeve is custom fit to the user and the locations of each of the one or more openings are custom positioned.

Another aspect of the present disclosure relates to a method of self-needling comprising providing device with one or more needle guides thereon wherein the device is fit to the user by a medical professional and the locations of the needle guides are customized to the user's anatomy. Needles are provided for insertion into the user's anatomy through the device and the needle guides by the user.

Yet another aspect of the present disclosure relates to a method of self-administering therapeutic dry needling comprising donning a sleeve on a selected body location and inserting one or more needles through one or more corresponding needle guides held by one or more corresponding apertures in the sleeve. The one or more needle guides are secured to one or more target locations on the sleeve wherein the target locations correspond to muscle locations on the selected location of the user such that the needles are inserted through the needle guide and into the selected muscle there below. The sleeve has dimensions specific to hold the sleeve and one or more needle guides on the selected location on the user while inserting the one or more needles through the one or more needle guides.

Separating the one or more needle guides from the sleeve allows for washing or sanitizing the one or more needle guides and/or the sleeve.

The sleeve and the one or more needle guides are comprised of different materials.

Self-administering dry needling further includes leaving the one or more needles inserted in the selected muscle through the one or more corresponding needle guides and protecting the one or more needles from unintentional movement or dislodgement by an upright surface of the one or more corresponding needle guides.

The needles may be retained penetrated into the muscle a selected period of time ranging from about 1 second to about 10 minutes.

### BRIEF DESCRIPTION OF THE DRAWINGS

FIG. 1 illustrates a system with a device according to one or more embodiments described herein.

FIGS. 2A-2D illustrate embodiments of needle guides of the device.

FIG. 3 illustrates another embodiment of the device.

FIG. 4 illustrates another embodiment of the device.

FIG. 5 illustrates another embodiment of the device.

FIG. 6 is a flow chart of a method of using a device according to one or more embodiments described herein.

### DETAILED DESCRIPTION

The devices described herein are to be worn by a user and provide a guide for self-needling therapy. The device is fit specifically for a user and can be selected by user based on location for use and/or by a medical professional. The alignment and orientation marks for each needle are marked on the device. Needle holders, or needle locators, are provided on the sleeve so that the user can place the needles into the holders to self-administer treatment.

The devices described herein can be provided in constructions configured for various locations on a user, examples of which include but are not limited to the forearm, shoulder (posterior, anterior, and lateral configurations), thigh (posterior, anterior, and lateral configurations), lower leg (posterior and anterior configurations), and foot.

The device for each location may be provided in one or more standard sizes, including a standard size and an extra large size.

The device may comprise one series of openings for needle holders or needle guides, or a plurality of series of openings for needle holders, allowing for customizable therapy and selected therapy and options for locations for needles. The device may be comprised of a material that allows for movement of the locations for one or more needle holders or other adjustment of location thereof. The needle holders may be removable from openings in the device configured to hold the needle holders securely therein. This may also allow for selected use and position of available needle holders.

In one or more embodiments, the device may be a sleeve. What is meant by a “sleeve” is that the device is configured to surround or otherwise fit securely around a target therapy location on the user (e.g., shoulder, forearm, leg etc.). The device may be slid on to a limb or other location on the user. In one or more embodiments, the device may be a unitary construction that is flexible and resilient and may be comprised of a material with a spandex content allowing for donning and snug fit.

Additionally, or alternatively the device may be a sleeve or sheet that is configured to wrap around the target therapy location and secured thereto with one or more closures or fasteners such as a clips, clasps, ties, hook and loop fastener (e.g., Velcro®) or other means configured to allow for adjusted fit.

The device and system described herein allows a user to be guided as to where and how to dry needle themselves for self-administered treatment. The user will don or otherwise apply the device to the selected body part or location suffering from pain or discomfort, needle their muscle, and resolve their pain at home. The device allows the user to dry needle their own muscles, generally without additional assistance. The device has one or more openings and one or more holders for a needle to be inserted. The holder indicates where a user should insert a needle(s) to help resolve their pain and discomfort. There are specific locations and/or trigger points that can be needled for the fastest pain relief or comfort results and the device has one or more openings/holders configured to correspond to these specific locations when worn. In addition, there are areas that should be avoided on a user and thus, there will not be openings that correspond to such locations. This allows a user to use the device with no significant risk of injury is used according to instructions. The device allows for safe, guided, access to self-treatment and allows for pain resolution that is quicker and a lower cost in comparison to seeing a clinician.

Devices and systems according to this disclosure remove accessibility issues. The device and system allow anyone to have access to treatment. The device allows for a lower cost for the user and it allows the user to perform self-needling at any time of the day and in their own home.

For example, to self-administer dry needling or otherwise self-needle comprises uses sanitizing wipes to clean and prep their skin. The device is applied to a selected location on the user's body. The device may fit around the selected body part and may provide visual cues on it for proper placement with openings/space location for dry needles to be inserted. An instructional sheet provided with use instructions for each specific body part may be included to give the user directions on how to apply the device as well as how to insert and use dry needles. Needles are inserted into the openings of the device and into the user's muscle.

Muscles that may be dry needled such as the forearm include locations for needle insertion for effective treatment of the area. The device may then have openings in this area for the needles to be inserted.

The device may be configured to fit onto a body part such as the forearm, shoulder, foot, thigh, or lower leg and may enclose or otherwise encircle the users body part. The device may provide a snug fit and may be provided in multiple sizes to select from, for example, a “one size fits most” and an “extra-large size” tailored for the contours and general anatomy of selected body parts including but not limited to those noted above. The user may wear the device and ensure sufficient placement for use with directions provided for correct placement. The device may be secured on the user via fasteners such as clips, hooks, clasps, hook and loop fastener or the device may comprise an elastic material in a content sufficient to allow the device to be slid on to the user and retained safely in place once worn and resistance to unintentional movement of the device.

Once the device is worn, the user may follow the instructional sheet on how to use dry needles and where to place the needles (the device helps to guide the user on where to place the needles). The device may have an opening/space with a needle holder that allows the user to insert the needle in the correct location and perform self-treatment. The device may have one or more specific openings/locations to insert the needle to effectively treat the body/muscle. Prior to donning the device, the user may prep their skin with a sanitation wipe to ensure it is clean.

The device may be able to fit most users and is able to expand but also fit snugly on or around a user's body part. The device supports one or more needle holders which act as a need guide for insertion of dry needles. The needle holder(s) may hold the needle vertically, and have an opening so the needle may then penetrate the user for insertion into the muscle of the user at a location identified by the location of the needle holder. The device is configured for use and to help guide the user on the best location to insert a needle.

To use this device, the device needs to fit onto one's body part, and a user may then follow the instructions for use and use the needles for treatment. The device helps guide the user on best location of needle placement.

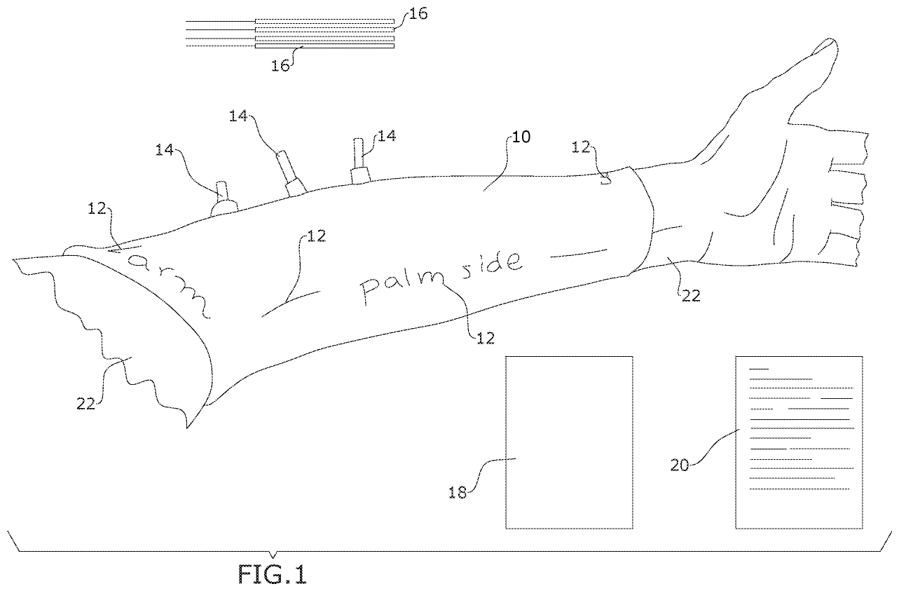

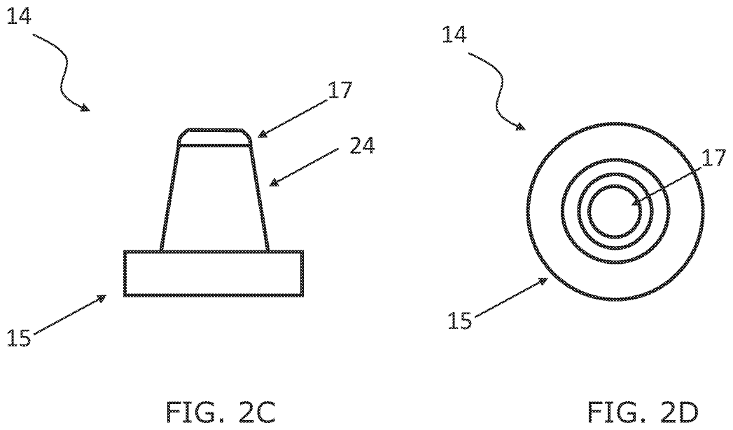

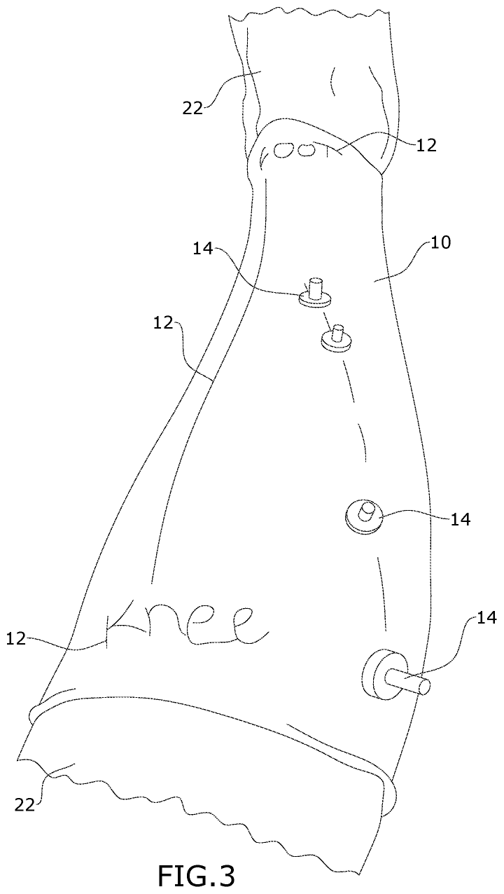

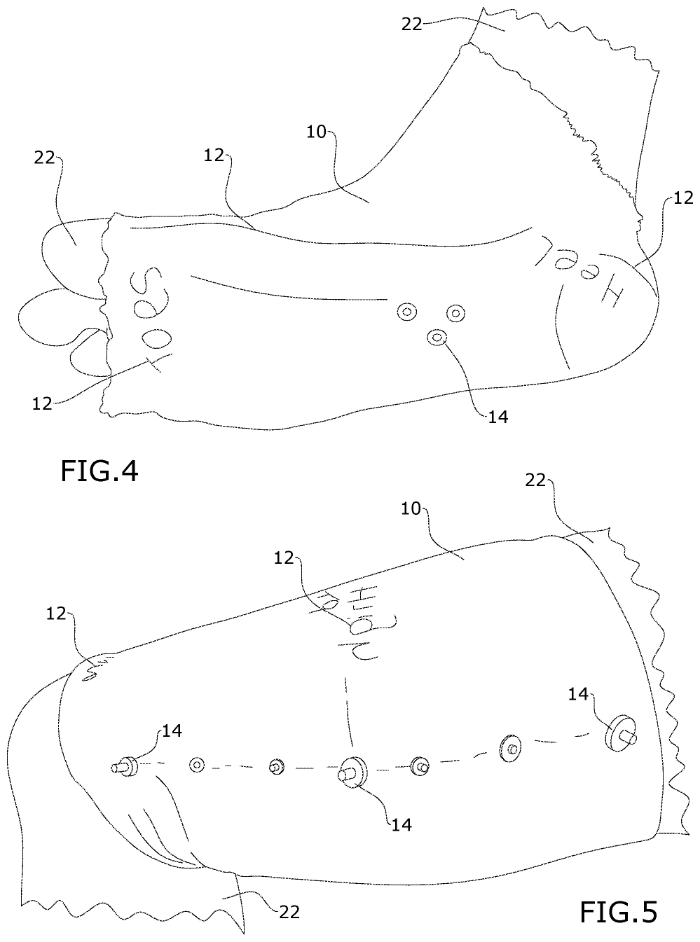

FIGS. 1-5 illustrate a sleeve 10 for wearing by a user and components. The sleeve 10 illustrated in worn on a limb 22. The sleeve comprises orientation or alignment marks 12 and a plurality of needle locators 14 which also act as holders for needles 16. In use a sanitizing wipe 18 is provided for cleaning the limb 22 prior to donning the sleeve 10 and instructions 20 for use may also be provided.

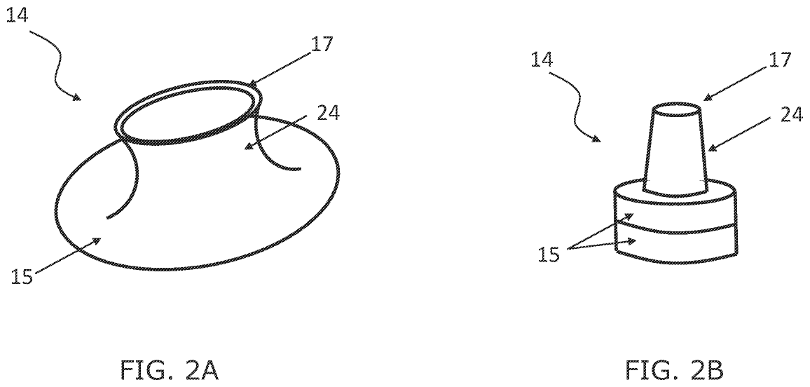

The needle locator 14 may generally be a port having a first end 15 for coupling to or otherwise being held by a location in the sleeve 10. The sleeve 10 may be provided with openings which frictionally engage the first end 15 of the needle locator 14. Alternatively, the first end 15 may have a lip or other ring which is provided between layers of the sleeve 10 construction or is otherwise sewn in or adhered to the sleeve 10. Further, the needle locator 14 may have a multi-piece first end 15 which allows the sleeve 10 to be secured between a lower and upper connection on the first end 15 as shown by the embodiment in FIG. 2B. In some embodiments, the sleeve 10 may hold the lip or ring of the first end 15 of the needle locator between the user's skin and the sleeve 10. The needle locators 14 may be removable for washing of the locators 14 and/or sleeve or may be permanently secured to the sleeve 10. A second end 17 of the needle locator 14 is configured for removably receiving the needle 16 and directing the needle into the location on the user. The needle locator 14 has an interior opening for directing and holding the needle. The needle locator 14 also has a length 24 there along which can prevent the needle 16 from excessive movement or dislodgement when the needle 16 is inserted into the user through the needle guide 14 and corresponding opening in the sleeve 10. The sleeve 10 thus allows a user access to selected guided locations with the needle 16 and retain the needles in the user's skin for a therapeutic effect.

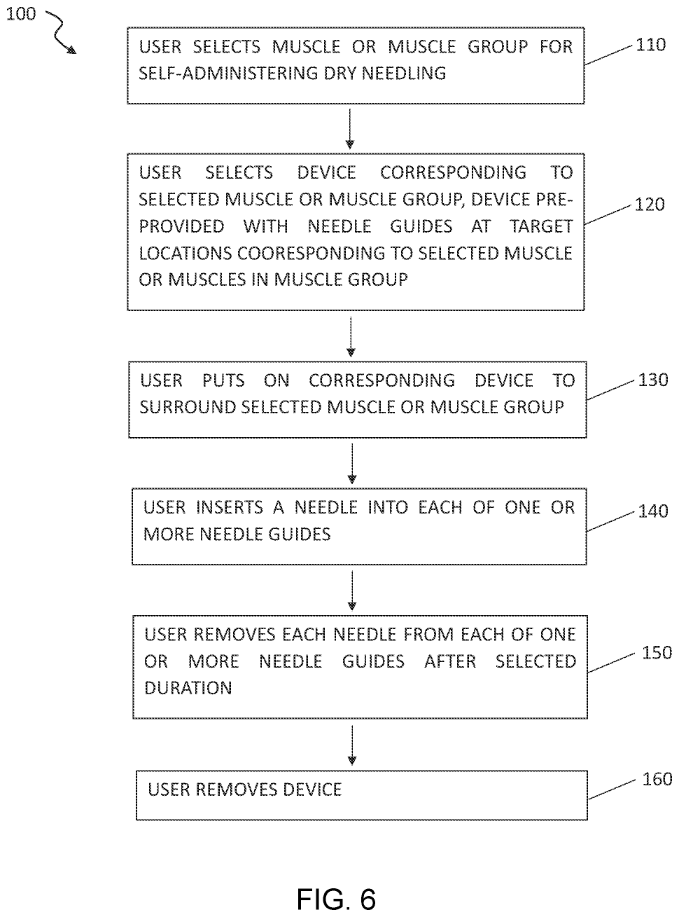

FIG. 6 illustrates one embodiment of a method 100 for self-administering dry needling therapy. For example, a user selects a muscle or muscle group for self-administering dry needling (110). The user then selects a device having dimensions and needle locator ports corresponding to the selected muscle(s) or muscle group (120) and puts on the corresponding device (130) to cover or surround the selected muscle(s) or muscle group. For example, when ready to dry needle, the user may sanitize the area first and then put the device on their limb or body. The user then inserts a needle through each of the one or more needle guides and into the selected muscle below the needle guide (140). The use may insert and then immediately remove the needles, allowing the needle to momentarily penetrate the muscle or may leave the inserted needle in the needle guide and in the muscle for a period of time ranging from about 1 second to about 10 minutes, or for example for about 4 minutes to about 8 minutes, or about 5 minutes, to achieve a therapeutic effect. The user then removes the device (160).

Although the present disclosure has been described with reference to preferred embodiments, workers skilled in the art will recognize that changes may be made in form and detail without departing from the spirit and scope of the disclosure.

1. A system for dry needling comprising:
a device comprising a surface area configured to surround a location on a user,
one or more openings in the surface area, each opening supporting a directional needle guide configured to allow a needle to pass therethrough to the location on the user; and
one or more needles configured to fit within each directional needle guide.

2. The system of claim 1 wherein the surface area comprises a flexible material, fabric, or plastic and is configured to removably surround the location on the user.

3. The system of claim 2 wherein the surface area is a sleeve configured to surround an arm, leg, or other appendage of the user.

4. The system of claim 1 wherein the openings in the surface area correspond to one or more muscle locations on the user such that when one of the one or more needles is inserted into each directional guide the needle is in a position to penetrate the user into the muscle tissue.

5. The system of claim 1 wherein the user is able to self-administer the needles.

6. The system of claim 3 wherein the sleeve is custom fit to the user and the locations of each of the one or more openings are custom positioned.

7. A method for self-administering therapeutic dry needling comprising:
donning a sleeve on a selected location on a user; and
inserting one or more needles through one or more corresponding needle guides held by one or more corresponding apertures in the sleeve into a selected muscle positioned therebelow,
wherein the one or more needle guides are secured to one or more target locations on the sleeve wherein the target locations correspond to muscle locations on the selected location of the user, and
wherein the sleeve has dimensions specific to hold the sleeve and one or more needle guides on the selected location on the user while inserting the one or more needles through the one or more needle guides and into the selected muscle.

8. The method of claim 7 and further comprising separating the one or more needle guides from the sleeve for washing or sanitizing the one or more needle guides and/or the sleeve.

9. The method of claim 7 wherein the sleeve and the one or more needle guides are comprised of different materials.

10. The method of claim 7 and further comprising leaving the one or more needles inserted in the selected muscle through the one or more corresponding needle guides and protecting the one or more needles from unintentional movement or dislodgement by an upright surface of the one or more corresponding needle guides.

11. The method of claim 10 and further comprising leaving the one or more needles inserted for a selected period of time ranging from about 1 second to about 10 minutes.
